(19) United States
(12) Patent Application Publication
(10) Pub. No.: US 2006/0263811 A1
(43) Pub. Date: Nov. 23, 2006

(54) Materials and kits for use in hot-start PCR, and methods of amplifying nucleic acids in a polymerase chain reaction

(76) Inventors: GeunSook Jeon, Foster City, CA (US); Charles S. Vann, Burlingame, CA (US); Achim E. Karger, Foster City, CA (US); Aldrich N. K. Lau, Palo Alto, CA (US); Shaheer H. Khan, Foster City, CA (US)

Correspondence Address: BRINKS HOFER GILSON & LIONE, P.O. BOX 10395, CHICAGO, IL, 60610, US

(21) Appl. No.: 11/416,784

(22) Filed: May 3, 2006

Related U.S. Application Data
(60) Provisional application No. 60/677,281, filed on May 3, 2005.

(51) Int. Cl. C12Q 1/68; C08G 63/91; C12P 19/34; C12N 9/22; C12M 1/34

(52) U.S. Cl. 435/6; 435/91.2; 435/199; 435/287.2; 525/54.2

(57) ABSTRACT

Materials for sequestering reagents in hot-start PCR, kits containing such materials, and methods for the use of such materials in amplifying nucleic acids are described.

### RELATED APPLICATIONS

This application claims the benefit of U.S. Provisional Application No. 60/677,281, filed May 3, 2005, the entire contents of which are incorporated by reference herein.

### FIELD

The present teachings relate to materials for sequestering PCR reagents in hot-start PCR, to methods of amplifying nucleic acids using such materials, and to kits containing such materials.

### INTRODUCTION

The polymerase chain reaction (PCR) has greatly advanced the field of molecular biology by allowing the amplification and analysis of specific fragments of DNA. However, PCR is prone to several types of artifacts that can frustrate analysis. For example, non-specific amplification of fragments may result from one or both of the primers binding to a sequence other than the target sequence, thereby producing one or more fragments of DNA that are not the desired product. Non-specific amplification (e.g., primer dimer and other undesired extension products) competes with amplification of the desired targets and decreases the overall efficiency of PCR. Moreover, amplification of non-specific products can occur even below ambient temperature.

### SUMMARY

The scope of the present invention is not intended to be limited by any of the statements within this summary.

In some embodiments, a material for use in hot-start PCR is provided that comprises a polylactone matrix and a PCR reagent. The polylactone matrix substantially sequesters the PCR reagent from participation in a polymerase chain reaction at ambient temperature.

In some embodiments, a material for use in hot-start PCR is provided that comprises (a) a polylactone matrix comprising a polylactone selected from the group consisting of poly(β-propiolactone), poly(β-butyrolactone), poly(γ-butyrolactone), poly(α-methyl-γ-butyrolactone), poly(γ-methyl-γ-hexanolactone), poly(γ-valerolactone), poly(γ-caprolactone), poly(δ-valerolactone), poly(ε-caprolactone), poly(η-heptanolactone), and combinations thereof; and (b) a PCR reagent selected from the group consisting of oligonucleotides, deoxynucleoside triphosphates, polymerase enzymes, metal ions, and combinations thereof. The polylactone matrix substantially sequesters the PCR reagent from participation in a polymerase chain reaction at ambient temperature.

In some embodiments, a method of amplifying nucleic acid in a polymerase chain reaction is provided. The method comprises (a) providing a target nucleic acid sequence; (b) combining the target nucleic acid sequence with a plurality of PCR reagents, wherein at least one of the plurality of PCR reagents is introduced in a polylactone matrix and is not available to participate in the polymerase chain reaction at ambient temperature; and (c) heating the polylactone matrix to release the at least one of the plurality of PCR reagents therein.

In some embodiments, a kit for hot-start PCR is provided. The kit comprises a material comprising a polylactone matrix and a first PCR reagent. The polylactone matrix substantially sequesters the first PCR reagent from participation in a polymerase chain reaction at ambient temperature.

These and other features of the present teachings are~set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 shows precursors to several representative polylactones in accordance with the present teachings.

FIG. 2 shows a photograph of an ethidium bromide-stained agarose gel obtained from a series of experiments described herein.

### DETAILED DESCRIPTION

It is to be understood that both the foregoing general description and the following detailed description are exemplary and explanatory only and are not restrictive of the invention. As used herein, the use of the singular (e.g., a PCR reagent, a polylactone, etc.) includes the plural unless specifically stated otherwise. In addition, the use of “or” means “and/or” unless stated otherwise. Furthermore, the use of the term “including” and various tenses thereof is not limiting.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety, except that in the event of any inconsistent disclosure or definition from the present application, the disclosure or definition herein shall be deemed to prevail.

Standard reference works setting forth general principles of PCR known to those of skill in the art include but are not limited to: Current Protocols in Molecular Biology, Ausubel et al. (John Wiley & Sons, New York, 2001); The Polymerase Chain Reaction, K. B. Mullis, F. Ferre, and R. A. Gibbs, Eds. (Birkhauser, Boston, 1994); and Molecular Cloning: A Laboratory Manual, 3^{rd} ed., J. Sambrook, & D. Russell, eds. (Cold Spring Harbor Laboratory Press, Cold Spring Harbor, N.Y., 2001).

By way of introduction, polylactone matrixes have been discovered whereby one or more components of PCR can be sequestered at ambient temperature (for example, by encapsulation and/or micro-encapsulation in a matrix) and then released at elevated temperature to effect PCR. It has been found that the sequestering of PCR components in these polylactone matrices reduces non-specific amplification occurring at ambient temperature and increases the efficiency of PCR. Thus, in some embodiments, more expensive PCR reagents such as AmpliTaq Gold® may be substituted with less expensive reagents, such as AmpliTaq®, while still achieving a reduction in undesired non-specific amplifications.

In accordance with the present teachings, as further described below, PCR reagents such as oligonucleotides, deoxynucleoside triphosphates (dNTPs), polymerase enzymes, metal ions, and the like, and combinations thereof, are encapsulated or micro-encapsulated in polymer beads, such as polylactone beads. The polymer is a solid at ambient temperature and melts at elevated temperature. In some embodiments, one or more PCR components can be added to the melted polymer to be encapsulated, thereby becoming isolated from other PCR reagents. The encapsulated components are substantially sequestered by the beads at ambient temperature and are not available to participate in PCR. Thus, actual PCR will not begin until the sequestered components are released into the reaction solution at elevated temperature during thermal cycling.

As further described below, the encapsulating polymer material does not interfere with PCR. Moreover, non-specific amplification, such as primer dimer and mispriming, can be reduced for hot-start PCR. In addition, the polymer beads containing the PCR component or components may be advantageously shipped at room temperature.

Most of the words used in this specification have meanings that would be attributed to those words by one skilled in the art. Words specifically defined in this specification have the meaning provided in the context of the present teachings as a whole, and as are typically understood by those skilled in the art. In the event that a conflict arises between an art-understood definition of a word or phrase and a definition of the word or phrase as specifically taught in this specification, the definition herein shall be deemed to prevail.

Throughout this description and in the appended claims, the following definitions are to be understood:

The phrases “PCR reagent,” “PCR component,” and the like refer to any chemical and/or biological entity required for the initiation, propagation, and/or termination of a polymerase chain reaction. By way of illustration, these phrases include but are not limited to: oligonucleotides; deoxynucleoside triphosphates, polymerase enzymes, metal ions, and the like, and combinations thereof.

The phrase “polylactone matrix” refers to a material containing a polylactone or-a mixture of two or more polylactones. The term “polylactone” refers to a polymer prepared from lactone monomers (e.g., via anionic polymerization, cationic polymerization, and the like). In some embodiments, polylactones are prepared by the ring opening polymerization of a lactone precursor, as shown in Eq. (1):

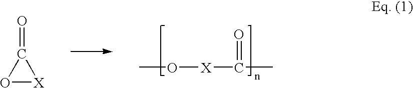

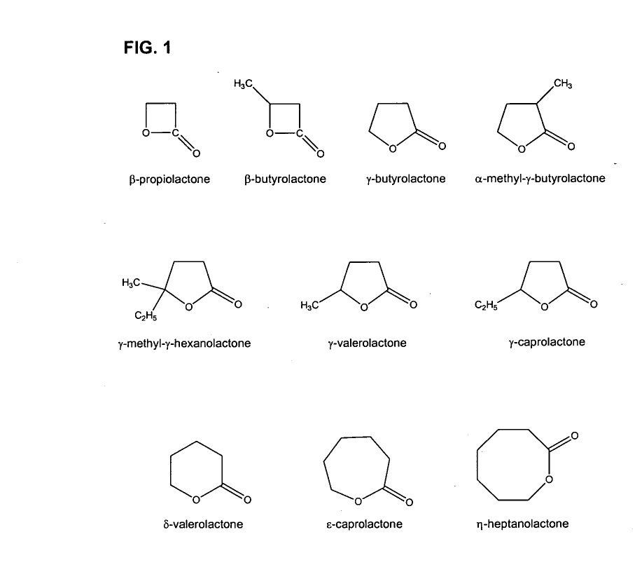

In Eq. (1), X represents a linear or branched alkylene residue (e.g., C_{y}H_{2y}, and branched derivatives thereof, wherein y is an integer ranging from 1 to about 20). An illustrative and non-comprehensive list of lactone precursors that can be used to form polylactones in accordance with the present teachings are shown in FIG. 1.

The term “sequestered” and various tenses thereof refers to the withholding of one or more PCR components from a polymerase chain reaction by retention within and/or by other physical or chemical agency of a polylactone matrix, such that progression of PCR is substantially minimized and/or entirely prevented in the absence of the sequestered component or components.

The term “encapsulated” refers to the distribution of one or more components throughout various portions of a matrix-forming material (e.g., one or more polylactones). By way of illustration, one or more PCR components can be “encapsulated” by a polylactone matrix by mixing these components with the polylactone in its molten state; thus, upon solidification of the polylactone matrix, the PCR components are distributed throughout various portions thereof.

The term “micro-encapsulated” refers to the enclosure of one or more components within an interior core portion of a matrix, whereby this interior core portion is substantially surrounded by an exterior shell portion formed substantially from the homogeneous matrix material (e.g., polylactone). By way of illustration, one or more PCR components can be “micro-encapsulated” by a polylactone matrix through various techniques known in the art, including but not limited to injection methods, inverse emulsion polymerization, surface spray-coating in a tumbler (pan-coating), or surface coating by fluidized bed.

The term “DNA” refers to deoxyribonucleic acid in its various forms as understood in the art, such as genomic DNA, cDNA, isolated nucleic acid molecules, vector DNA, chromosomal DNA, and the like. The phrase “nucleic acid” refers to DNA or RNA in any form. Examples of isolated nucleic acid molecules include but are not limited to recombinant DNA molecules contained in a vector, recombinant DNA molecules maintained in a heterologous host cell, partially or substantially purified nucleic acid molecules, synthetic DNA molecules, and the like.

The phrase “non-specific amplification” refers to amplification of a region of a nucleic acid that is not the portion of the nucleic acid that is the target nucleic acid, and includes primer dimer formation and other extension products. By way of illustration, examples of non-specific amplification include but are not limited to amplification of a region of DNA that is unrelated to the target sequence; amplification of a related DNA sequence, but from a different region of DNA than that targeted for amplification; amplification of the target sequence but comprising more or less nucleobases than the intended amplified fragment due to inexact annealing of at least one primer to the target sequence; and the like.

The term “anneal” and various tenses thereof refers to specific interaction between strands of nucleotides wherein the strands bind to one another substantially based on complementarity between the strands as determined by Watson-Crick base pairing. It is not necessary that complementarity be 100% for annealing to occur.

The term “amplification” and various tenses thereof refer to enzymatically increasing the amount of a specific nucleotide sequence in a polymerase chain reaction.

The term “incubating” and various tenses thereof refers to a maintaining a state of controlled conditions, such as temperature, over a period of time.

The term “denaturation” and various tenses thereof refer to the separation of nucleotide strands from an annealed state. Denaturation can be induced by a number of factors including but not limited to ionic strength of the buffer: temperature: chemicals that disrupt base pairing interactions; and the like.

The phrase “sufficient amount of time” used in reference to time for the amplification of nucleic acid refers to the time which allows the enzyme or enzymes used to complete the polymerization of deoxynucleoside triphosphates into the amplifying nucleic acid. The amount of time required varies depending on several factors which are well-known by persons of ordinary skill in the art.

The phrase “conditions sufficient to amplify” refers to reaction conditions for the PCRs. The reaction conditions include the chemical components of the reaction, the temperatures used in the reaction cycles, the number of cycles of the reaction, and the time of the stages of the reaction cycles.

Materials for use in hot-start PCR embodying features of the present teachings comprise (a) a polylactone matrix, and (b) one or a plurality of PCR reagents. In this material, the polylactone matrix substantially sequesters the PCR reagent from participation in a polymerase chain reaction at ambient temperature. In some embodiments, one or a plurality of PCR reagents is encapsulated by the polylactone matrix, whereas in other embodiments, one or a plurality of PCR reagents is micro-encapsulated by the polylactone matrix.

Materials in accordance with the present teachings can have any manner of shape in the solid state, including all manner of regular and irregular geometric shapes. Representative shapes includes but are not limited to spherical, discs or wafers (e.g., having cross-sections that are circular, elliptical, square, rectangular, triangular, spherical triangular, or the like), hemispheres, spherical cones, ellipsoids, oblate spheroids, prolate spheroids, catenoids, spherical lunes, spherical wedges, cones, cylinders, truncated cylinders, ungula of cylinders, quoits, toroids, zones of spheres, parallelepipeds, cubes, tetrahedrons, bispheonids, pyramids, and the like. In some embodiments, the solid material comprises a substantially spherical bead shape.

The dimensions of a solid-state material in accordance with the present teachings (e.g., the average diameter of a substantially spherical bead) are not limited, and can be varied to provide materials having dimensions appropriate for addition to particular types of reaction vessels (e.g., eppendorf tubes, etc.). Similarly, the weights of a solid-state material (e.g., the average weight of a substantially spherical bead) are likewise not limited, and can be varied according to the desired final concentration of PCR components to be added to a reaction mixture. For example, smaller bead weights can be employed when the concentration of PCR components in the bead is high, whereas larger weights can be used when the concentration of PCR components in the bead is low. Alternatively, to increase the final concentration of one or more PCR components in a reaction vessel, a larger number of individual units (e.g., spherical beads) can be added to the reaction mixture.

Materials in accordance with the present teachings can be delivered to PCR reaction vessels by all manner of manual or automated delivery, and may be manipulated in accordance with methods and devices described in U.S. Patent Application Publication Nos. US 2004/0086426 A1 and US 2003/0205200 A1, both assigned to the assignee of the present invention.

The polylactone matrix used in accordance with the present teachings can be formed from a single material or from a mixture of different materials. In some embodiments, the polylactone matrix comprises one or a plurality of polylactones. Representative polylactones for use in accordance with the present teachings include but are not limited to poly(β-propiolactone), poly(β-butyrolactone), poly(γ-butyrolactone), poly(α-methyl-γ-butyrolactone), poly(γ-methyl-γ-hexanolactone), poly(γ-valerolactone), poly(γ-caprolactone), poly(δ-valerolactone), poly(ε-caprolactone), poly(η-heptanolactone), and combinations thereof. In some embodiments, the polylactone is selected from poly(β-propiolactone), poly(γ-butyrolactone), poly(δ-valerolactone), poly(ε-caprolactone), poly(η-heptanolactone), and combinations thereof. In some embodiments, the polylactone matrix comprises poly(ε-caprolactone).

The type of PCR reagent sequestered by the polylactone matrix is not limited, and includes any reagent and other chemical and/or biological entity used in a polymerase chain reaction. Representative PCR reagents for use in accordance with the present teachings include but are not limited to oligonucleotides, dNTPs, polymerase enzymes, metal ions, and combinations thereof.

Representative oligonucleotides for use in accordance with the present teachings include but are not limited to antisense primers, sense primers, and a combination thereof. In some embodiments, oligonucleotide primers are added to the reaction and demarcate the 5′ and 3′ ends of the amplified fragment. One oligonucleotide primer anneals to the sense (+strand) of the denatured template DNA, and the other oligonucleotide primer anneals to the antisense (−strand) of the denatured template DNA. In some embodiments, oligonucleotide primers are 12-25 nucleotides in length; however, they may be shorter or longer depending on the specific template sequence to be amplified, and the length of the primer is not limited. Oligonucleotide primers can be designed to anneal to specific portions of a nucleic acid that flank a target of interest so as to specifically amplify the portion of DNA between the primers complementary sites. In some embodiments, oligonucleotide primers are chemically synthesized. One of ordinary skill in the art can easily design specific primers to amplify a target of interest. Furthermore, there are many known primer sequences for amplification, and any of these may be used in accordance with the present teachings.

Oligonucleotide primers in accordance with the present teachings may be composed of adenosine, thymidine, guanosine, cytidine, uridine, nucleoside analogs (e.g., locked nucleic acids (LNA), peptide nucleic acid (PNA), phosporamidites), and nucleosides containing or conjugated to chemical moieties such as radionuclides (e.g., ³²P, ³⁵S), fluorescent molecules, minor groove binders, or any other nucleoside conjugate known in the art.

In some embodiments, a fluorophore can be used to tag at least one primer of the PCR reaction. In some embodiments primers for different target fragments can be tagged with different fluorophores (that produce differently colored products) and may be used in the same multiplex PCR reaction and subsequently analyzed together. In some embodiments, the forward primer is tagged, but the reverse primer may also be tagged. Examples of fluorophores include but are not limited to fluorescein (which absorbs maximally at 492 nm and emits maximally at 520 nm); TAMRA, N,N,N′,N′-tetramethyl-6-carboxyrhodamine (which absorbs maximally at 555 nm and emits maximally at 580 nm); FAM, 5-carboxyfluorescein (which absorbs maximally at 495 nm and emits maximally at 525 nm); JOE, 2′,7′-dimethoxy-4′,5′-dichloro-6-carboxyfluorescein (which absorbs maximally at 525 nm and emits maximally at 555 nm); ROX, 6-carboxy-X-rhodamine (which absorbs maximally at 585 nm and emits maximally at 605 nm); CY3 (which absorbs maximally at 552 nm and emits maximally at 570 nm); CY5 (which absorbs maximally at 643 nm and emits maximally at 667 nm); TET, tetrachloro-fluorescein (which absorbs maximally at 521 nm and emits maximally at 536 nm); and HEX, hexachloro-fluorescein (which absorbs maximally at 535 nm and emits maximally at 556 nm).

Deoxynucleotide triphosphates are the building blocks of the amplifying nucleic acid molecules. Representative dNTPs for use in accordance with the present teachings include but are not limited to deoxyadenosine triphosphate (dATP), deoxyguanosine triphosphate (dGTP), deoxycytidine triphosphate (dCTP) and thymidine triphosphate (dTTP), and combinations thereof. Other dNTPs, such as deoxyuridine triphosphate (dUTP), deoxyinosine triphoshpate (dITP), dNTP analogs, and conjugated dNTPs may also be used, and are encompassed by the term dNTPs as used herein.

The polymerase enzymes in accordance with the present teachings that polymerize the nucleotide triphosphates into the amplified fragments of the PCR may be any polymerase known in the art, including but not limited to heat-stable DNA polymerases. Representative enzymes include but are not limited to DNA polymerase from organisms such as Thermus aquaticus, Thermus thermophilus, Thermococcus litoralis, other Thermus species, Bacillus stearothermophilus, other Bacillus species, Thermococcus species, Thermotoga maritime, other Thermotoga species, and Pyrococcus species. In some embodiments, the heat-stable DNA polymerase comprises a Taq DNA polymerase.

The polymerase enzyme used in accordance with the present teachings may be isolated from the bacteria, produced by recombinant DNA technology or purchased from commercial sources. By way of illustration, DNA polymerases are available from Applied Biosystems (Foster City, Calif.) and include but are not limited to AmpliTaq Gold® DNA polymerase; AmpliTaq® DNA Polymerase; AmpliTaq® DNA Polymerase, Stoffel fragment; rTth DNA Polymerase; and rTth DNA Polymerase XL. Other suitable polymerases include but are not limited to Tne, Bst DNA polymerase large fragment from Bacillus stearothermophilus; Vent and Vent Exo- from Thermococcus litoralis; Tma from Thermotoga maritima; Deep Vent and Deep Vent Exo- and Pfu from Pyrococcus; and mutants, variants and derivatives thereof.

Metal ions (e.g., divalent metal ions) are often advantageous to allow the polymerase to function efficiently. Representative metal ions for use in accordance with the present teachings include but are not limited to magnesium, manganese, calcium, cobalt, nickel, copper, zinc, iron, and combinations thereof. For example, but not by way of limitation, magnesium ion allows certain DNA polymerases to function effectively. In some embodiments, MgCl₂ or MgSO₄ is added to reaction buffers to supply the optimum magnesium ion concentration. In accordance with the present teachings, the magnesium source may be provided in a polylactone matrix, as further described below.

The magnesium ion concentration required for optimal PCR amplification may depend on the specific set of primers and template used. Thus, the amount of magnesium salt added to achieve optimal amplification is often determined empirically, and is a routine practice in the art. Generally, the concentration of magnesium ion for optimal PCR can vary between about 1 and about 10 mM. In some embodiments, a range of magnesium ion concentration in PCR reactions is between about 1.0 and about 4.0 mM, varying around a midpoint of about 2.5 mM. However, the concentration of magnesium ion is not restricted.

In some embodiments, methods of amplifying nucleic acid in a polymerase chain reaction are provided. The methods comprise (a) providing a target nucleic acid sequence; (b) combining the target nucleic acid sequence with a plurality of PCR reagents, wherein at least one of the plurality of PCR reagents is introduced in a polylactone matrix and is not available to participate in the polymerase chain reaction at ambient temperature; and (c) heating the polylactone matrix to release the at least one of the plurality of PCR reagents therein.

PCR reaction time, temperatures, and cycle numbers can be varied to optimize a particular reaction as a matter of routine experimentation. Those of ordinary skill in the art will recognize the following as guidance in determining the various parameters for PCR reactions, and also will recognize that variation of one or more conditions is within the scope of the present teachings.

PCR reaction temperature and time is determined in three stages: denaturation, annealing, and extension. One round of denaturation, annealing, and extension is referred to as a “cycle.” Denaturation is generally conducted at a temperature that permits the strands of DNA to separate without destroying the activity of the polymerase. Generally, thermostable polymerases are used. However, heat-labile polymerases may alternatively be used if they are replenished after the denaturation step of the PCR. Thermostable polymerases can withstand high temperatures and maintain some level of activity. In some embodiments, denaturation is conducted above 90° C. and below 100° C. In some embodiments, denaturation is conducted at a temperature of 94-95° C. In some embodiments, denaturation of DNA is conducted for at least 1 to 30 seconds. In some embodiments, denaturation is conducted for 1 to 15 seconds. In other embodiments, denaturation is conducted for up to 1 minute or more. In addition to the denaturation of DNA, for some polymerases, such as AmpliTaq Gold®, incubation at the denaturation temperature also serves to activate the enzyme. Therefore, it may be advantageous to allow the first step of PCR (denaturation) to be longer than subsequent denaturation steps when these enzymes are used.

During the annealing phase, oligonucleotide primers anneal to the target DNA in their regions of complementarity and are substantially extended by the DNA polymerase once the latter has bound to the primer-template duplex.

In a conventional PCR, the annealing temperature typically is at or below the melting point (Tₘ) of the least stable primer-template duplex, where Tₘ can be estimated by any of several theoretical methods well known to practitioners of the art. For example, the Tₘ can be determined by Eq. (2):
Tₘ=(4° C.×# of G and C bases)+(2° C.×# of A and T bases) Eq. (2)

In some embodiments, the annealing temperature is 5° C. to 10° C. below the estimated Tₘ of the least stable primer-template duplex. In some embodiments, the annealing time is between about 30 seconds and about 2 minutes. However, in some embodiments, the annealing step is performed for a longer period of time than would be used in standard PCR protocols.

In some embodiments, the annealing phase is followed by an extension phase. “Extension” is conducted for a sufficient amount of time to allow the enzyme to complete primer extension into the appropriately sized fragments.

The number of cycles of PCR (denaturation, annealing and extension) used will determine the amount of amplification. PCR is an exponential amplification of DNA molecules. Thus, theoretically, after each cycle of PCR, there are twice the number of fragments that were present in the prior cycle. In some embodiments, 20-30 cycles of PCR are performed. In some embodiments, 25-30 cycles are performed, although cycle number is not particularly limited.

For some embodiments, it is advantageous to incubate the reactions at a certain temperature following the last phase of the last cycle of PCR. In some embodiments, a prolonged extension phase is selected. In other embodiments, an incubation at a low temperature (e.g., 4° C.) is selected.

In some embodiments, PCR is performed in the presence of sorbitol, or sorbitol and a denaturant, such as dimethyl sulfoxide (DMSO) to increase the yield of specifically amplified target DNA sequences, such as ribosomal DNA sequences (e.g., see U.S. Pat. No. 6,783,940, assigned to the assignee of the present invention).

The addition of a denaturant to PCRs may also increase specific target yield. Denaturants suitable for use in accordance with the present teachings include but are not limited to DMSO, 2-pyrrolidinine, and 1-methyl-2-pyrrolidinone. Other denaturants known in the art can also be used.

In some embodiments, kits for hot-start PCR are provided that comprise a material comprising a polylactone matrix and a first PCR reagent. In this material, the polylactone matrix substantially sequesters the first PCR reagent from participation in a polymerase chain reaction at ambient temperature.

The first PCR reagent in the polylactone matrix can comprise a single reagent or a plurality of reagents. Representative first PCR reagents for use in accordance with the present teachings include but are not limited to oligonucleotides, deoxynucleoside triphosphates, polymerase enzymes, metal ions, and combinations thereof. One or more additional reagents for PCR that are not already provided in the polylactone matrix (i.e., are not already provided as the “first PCR reagent”) can be separately provided in the kit (i.e., as a “second PCR reagent,” wherein the phrase “second PCR reagent” includes both single reagents and pluralities of reagents, provided either as individually packaged components and/or in combination).

As used herein, the term “kit” refers to an assembly of materials that are used in performing a method embodying features of the present teachings. The reagents may be provided in packaged combination in the same or in separate containers, depending on their cross-reactivities and stabilities, and in liquid or in lyophilized form. The amounts and proportions of reagents provided in the kit may be selected so as to provide optimum results for a particular application.

Reagents included in kits embodying features of the present teachings may be supplied in all manner of containers such that the activities of the different components are substantially preserved, while the components themselves are not substantially adsorbed or altered by the materials of the container. Suitable containers include but are not limited to ampoules, bottles, test tubes, vials, flasks, syringes, bags and envelopes (e.g., foil-lined), and the like. The containers may be formed of any suitable material including but not limited to glass, organic polymers (e.g., polycarbonate, polystyrene, polyethylene, etc.), ceramic, metal (e.g., aluminum), metal alloys (e.g., steel), cork, and the like. In addition, the containers may contain one or more sterile access ports (e.g., for access via a needle), such as may be provided by a septum. Preferred materials for septa include rubber and polymers including but not limited to, for example, polytetrafluoroethylene of the type sold under the trade name TEFLON by DuPont (Wilmington, Del.). In addition, the containers may contain two or more compartments separated by partitions or membranes that can be removed to allow mixing of the components.

Kits in accordance with the present teachings may also be supplied with other items known in the art and/or which may be desirable from a commercial and user standpoint, such as instructions for performing PCR, other known components of PCR reactions including but not limited to: solvents; buffers; detergents (e.g., Triton X-100, Nonidet P40 (NP-40), Tween-20) and agents that disrupt mismatching of nucleotide pairs, such as dimethylsulfoxide (DMSO), and tetramethylammonium chloride (TMAC); empty syringes; tubing, gauze, pads, disinfectant solution, etc.

In some embodiments, the solvents used for PCR contain a buffering agent, (e.g., Tris-HCl) and non-buffering salts (e.g., KCl). The buffering agent may be any known buffers in the art, and may be varied to optimize PCR results by routine experimentation. Persons of ordinary skill in the art will readily be able to determine optimal buffering conditions. Some PCR buffers may be optimized depending on the enzyme used. By way of illustration, AmpliTaq Gold® DNA polymerase has an optimum KCl concentration of 50 mM; AmpliTaq® DNA Polymerase, Stoffel fragment has an optimum KCl concentration of 10 mM; and rTth DNA Polymerase and rTth DNA Polymerase XL have an optimum KCl concentration of 75-100 mM.

Instructional materials provided with kits embodying features of the present invention may be printed (e.g., on paper) and/or supplied in an electronic-readable medium (e.g., floppy disc, CD-ROM, DVD-ROM, zip disc, videotape, audio tape, etc.). Alternatively, instructions may be provided by directing a user to an Internet web site (e.g., specified by the manufacturer or distributor of the kit) and/or via electronic mail.

PCRs performed in accordance with the present teachings may also be performed in the presence of other reagents to optimize amplification, which may optionally be provided in the above-described kits. By way of illustration, uracil N-glycosylase (UNG), such as included in the GeneAmp® PCR Carry-over Prevention Kit may be used. UNG may be included in the PCR reaction as an initial step to ensure that PCR products cannot be reamplified in subsequent PCR amplifications. The principle is based on an enzymatic reaction analogous to the restriction-modification and excision-repair systems of cells. PCR products from previous PCR amplifications in which dUTP has been incorporated are degraded. Native nucleic acid templates are unaffected. The method involves substituting dUTP for dTTP in the PCR mixture, and pretreating all subsequent PCR mixtures with the uracil N-glycosylase enzyme prior to PCR amplification. Uracil is excised from initial products using UNG and is eliminated by degrading the resulting abasic polynucleotide with heat.

Aspects of the present teachings may be further understood in light of the following representative procedures and examples, which are provided solely by way of illustration and should not be construed as limiting in any way the scope of the appended claims or their equivalents.

### EXAMPLES

The bead material used in the following representative examples was polycaprolactone, [—O(CH₂)₅CO]ₙ, which was purchased from Aldrich Chemical Co. (Milwaukee, Wis., Catalog #44,075-2). This material has an average molecular weight of ca 14,000, is water insoluble, and is a solid at room temperature (mp=60° C.).

Encapsulation of MgCl₂ in Polycaprolactone

187 mg of polycaprolactone in an eppendorf tube was melted at 80° C. in a water-contained heating block. 4.9 M MgCl₂ (10 μL, 13 mg) was added to the melted polymer at 80° C. The mixture was stirred well with a glass rod to make a homogeneous solution while maintaining the same temperature. An optical fiber was dipped into the solution and taken out to form a bead at the tip of the optical fiber. These beads were washed with water and then methanol and dried in the air. The weight of beads ranged from 1 mg (0.245 pmole of MgCl₂) to 5 mg (1.2 μmole of MgCl₂).

PCR Experiments

Each 100 μl PCR-reaction contained 10 μl PCR buffer II (no Mg) (from AmpliTaq® kit, Applied Biosystems #N8010055), 1 mM dNTPs (250 mM each dNTP, from Applied Biosystems #N8080261), 5 ng human gDNA (Applied Biosystems #403062A), 5 μl TaqMan™ RNase P Detection Reagents (Fam) (AB #4316831), 1.25 U AmpliTaq® (from AmpliTaq kit, Applied Biosystems #N8010055).

Mg was added to the reactions in one of two ways: either by adding MgCl₂ solution at various concentrations resulting in a final MgCl₂ concentration of 0-20 mM, or by adding a Mg-encapsulated polycaprolactone bead. Beads contained varying amounts of Mg which, assuming complete release, will result in final Mg concentrations of 3.92 to 5.4 to 9.06 mM Mg⁺⁺. A control reaction was set up with a straight (i.e., no Mg) polycaprolactone bead; Mg⁺⁺ was supplied to this reaction in the form of MgCl₂ solution to a 2.5 mM final concentration.

All reactions were in a 100 μl final volume. After reaction set-up, all reactions were incubated at 25° C for 3 hours prior to PCR temperature cycling emulating dwell time between reaction set-up and heating, which has been identified as an opportunity for unwanted pre-PCR mispriming and primer-dimer formation.

The human Rnase P gene was amplified from human genomic DNA by holding the reactions for 5 min at 95° C., and subsequently 40 cycles: 5 s at 96° C., 2 minutes at 60° C. All PCR reactions were analyzed by loading 10 μl into each well of 4% E-gel (Invitrogen) and performing a 30 min separation.

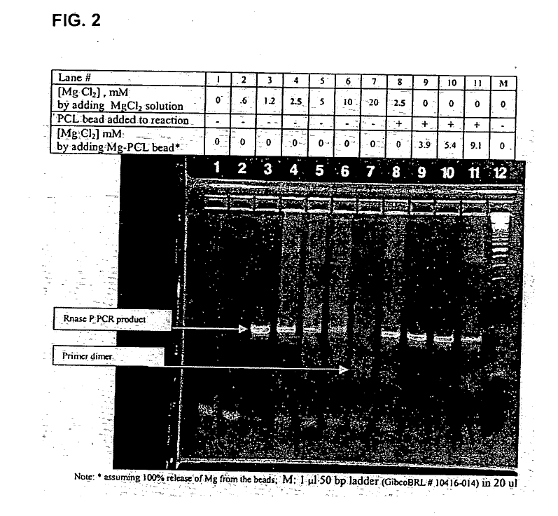

FIG. 2 shows the resulting gel from the above-described experiments after Ethidium Br photography. The gel image confirms that the Mg-encapsulated polycaprolactone beads convey hot-start properties to the PCR reaction. For the samples containing Mg introduced by adding MgCl₂ solution, the amount of product varied considerably with Mg concentration: essentially no product formed for 0 and 0.6 mM Mg; maximum product formation was observed for 1.2 mM Mg; and reduced product formation was observed for samples containing ≧5 mM Mg. Most of these conventional reactions showed some degree of primer dimer formation and unspecific priming with the least amount of undesired product being observed for 1.2 mM Mg, the concentration which also showed the highest yield of PCR product. A comparison of two reactions with the same 2.5 mM Mg concentration, with and without a straight (i.e., no Mg) polycaprolactone bead (lanes #4 and 8) shows that the straight polycaprolactone polymer has no inhibitory effect on PCR but does not show any beneficial effect in terms of diminished formation of unspecific product.

Comparing the reactions containing the Mg-encapsulated polycaprolactone beads to the conventional PCR reactions, it is evident that primer dimer and unspecific product is reduced considerably in the former, most likely due to the Mg only being made available after hot-start. Similar to standard reactions in which Mg was added as a solution, there is diminished product formation at very high Mg concentrations of about 10 mM and more in reactions where Mg is released from beads (see lanes #7 and 11).

In summary, as shown in FIG. 2, the polylactone beads in accordance with the present teachings release MgCl₂ at elevated temperature during PCR, and the polymeric material does not interfere with the PCR. Furthermore, reduction of primer dimer formation was observed.

The foregoing detailed description and accompanying drawings have been provided by way of explanation and illustration, and are not intended to limit the scope of the invention. Many variations in the presently preferred embodiments illustrated herein will be readily apparent to one of ordinary skill in the art, and remain within the scope of the invention.

1. A material for use in hot-start PCR comprising:
a polylactone matrix; and
a PCR reagent;
wherein the polylactone matrix substantially sequesters the PCR reagent from participation in a polymerase chain reaction at ambient temperature.

2. The material of claim 1 wherein the polylactone matrix comprises a polylactone selected from the group consisting of poly(β-propiolactone), poly(β-butyrolactone), poly(γ-butyrolactone), poly(α-methyl-γ-butyrolactone), poly(γ-methyl-γ-hexanolactone), poly(γ-valerolactone), poly(γ-caprolactone), poly(δ-valerolactone), poly(ε-caprolactone), poly(η-heptanolactone), and combinations thereof.

3. The material of claim 1 wherein the polylactone matrix comprises a polylactone selected from the group consisting of poly(β-propiolactone), poly(γ-butyrolactone), poly(δ-valerolactone), poly(ε-caprolactone), poly(η-heptanolactone), and combinations thereof.

4. The material of claim 1 wherein the polylactone matrix comprises poly(ε-caprolactone).

5. The material of claim 1 wherein the PCR reagent is selected from the group consisting of oligonucleotides, deoxynucleoside triphosphates, polymerase enzymes, metal ions, and combinations thereof.

6. The material of claim 5 wherein the oligonucleotides are selected from the group consisting of antisense primers, sense primers, and a combination thereof.

7. The material of claim 5 wherein the deoxynucleoside triphosphates are selected from the group consisting of dATP, dTTP, dCTP, dGTP, dUTP, dITP, and combinations thereof.

8. The material of claim 5 wherein the polymerase enzymes comprise a heat-stable DNA polymerase.

9. The material of claim 8 wherein the heat-stable DNA polymerase comprises an enzyme from a species selected from the group consisting of Thermus, Bacillus, Thermococcus, Thermotoga, Pyrococcus, and combinations thereof.

10. The material of claim 9 wherein the Thermus is selected from the group consisting of Thermus aquaticus, Thermus thermophilus, and a combination thereof.

11. The material of claim 9 wherein the Bacillus comprises Bacillus stearothermophilus.

12. The material of claim 9 wherein the Thermococcus comprises Thermococcus litoralis.

13. The material of claim 9 wherein the Thermotoga comprises Thermotoga maritama.

14. The material of claim 9 wherein the heat-stable DNA polymerase comprises a Taq DNA polymerase.

15. The material of claim 5 wherein the metal ions are selected from the group consisting of magnesium, manganese, calcium, cobalt, nickel, copper, zinc, iron, and combinations thereof.

16. The material of claim 5 wherein the metal ions comprise magnesium.

17. The material of claim 1 wherein the PCR reagent is encapsulated by the polylactone matrix.

18. The material of claim 1 wherein the PCR reagent is micro-encapsulated by the polylactone matrix.

19. A material for use in hot-start PCR comprising:
a polylactone matrix comprising a polylactone selected from the group consisting of poly(β-propiolactone), poly(β-butyrolactone), poly(γ-butyrolactone), poly(α-methyl-γ-butyrolactone), poly(γ-methyl-γ-hexanolactone), poly(γ-valerolactone), poly(γ-caprolactone), poly(δ-valerolactone), poly(ε-caprolactone), poly(η-heptanolactone), and combinations thereof; and
a PCR reagent selected from the group consisting of oligonucleotides, deoxynucleoside triphosphates, polymerase enzymes, metal ions, and combinations thereof;
wherein the polylactone matrix substantially sequesters the PCR reagent from participation in a polymerase chain reaction at ambient temperature.

20. The material of claim 19 wherein:
the oligonucleotides are selected from the group consisting of antisense primers, sense primers, and a combination thereof;
the deoxynucleoside triphosphates are selected from the group consisting of dATP, dTTP, dCTP, dGTP, dUTP, dITP, and combinations thereof;
the polymerase enzymes comprise a heat-stable DNA polymerase; and
the metal ions are selected from the group consisting of magnesium, manganese, calcium, cobalt, nickel, copper, zinc, iron, and combinations thereof.

21. The material of claim 20 wherein the PCR reagent is encapsulated by the polylactone matrix.

22. The material of claim 20 wherein the PCR reagent is micro-encapsulated by the polylactone matrix.

23. A method of amplifying nucleic acid in a polymerase chain reaction comprising:
(a) providing a target nucleic acid sequence;
(b) combining the target nucleic acid sequence with a plurality of PCR reagents, wherein at least one of the plurality of PCR reagents is introduced in a polylactone matrix and is not available to participate in the polymerase chain reaction at ambient temperature; and
(c) heating the polylactone matrix to release the at least one of the plurality of PCR reagents therein.

24. The method of claim 23 wherein the polylactone matrix comprises a polylactone selected from the group consisting of poly(β-propiolactone), poly(β-butyrolactone), poly(γ-butyrolactone), poly(α-methyl-γ-butyrolactone), poly(γ-methyl-γ-hexanolactone), poly(γ-valerolactone), poly(γ-caprolactone), poly(δ-valerolactone), poly(ε-caprolactone), poly(η-heptanolactone), and combinations thereof.

25. The method of claim 23 wherein the polylactone matrix comprises a polylactone selected from the group consisting of poly(β-propiolactone), poly(γ-butyrolactone), poly(δ-valerolactone), poly(ε-caprolactone), poly(η-heptanolactone), and combinations thereof.

26. The method of claim 23 wherein the polylactone matrix comprises poly(ε-caprolactone).

27. The method of claim 23 wherein the PCR reagents are selected from the group consisting of oligonucleotides, deoxynucleoside triphosphates, polymerase enzymes, metal ions, and combinations thereof.

28. The method of claim 27 wherein the oligonucleotides are selected from the group consisting of antisense primers, sense primers, and a combination thereof.

29. The method of claim 27 the deoxynucleoside triphosphates are selected from the group consisting of dATP, dTTP, dCTP, dGTP, dUTP, dITP, and combinations thereof.

30. The method of claim 27 wherein the polymerase enzymes comprise a heat-stable DNA polymerase.

31. The method of claim 30 wherein the heat-stable DNA polymerase comprises an enzyme from a species selected from the group consisting of Thermus, Bacillus, Thermococcus, Thermotoga, Pyrococcus, and combinations thereof.

32. The method of claim 31 wherein the Thermus is selected from the group consisting of Thermus aquaticus, Thermus thermophilus, and a combination thereof.

33. The method of claim 31 wherein the Bacillus comprises Bacillus stearothermophilus.

34. The method of claim 31 wherein the Thermococcus comprises Thermococcus litoralis.

35. The method of claim 31 wherein the Thermotoga comprises Thermotoga maritama.

36. The method of claim 31 wherein the heat-stable DNA polymerase comprises a Taq DNA polymerase.

37. The method of claim 27 wherein the metal ions are selected from the group consisting of magnesium, manganese, calcium, cobalt, nickel, copper, zinc, iron, and combinations thereof.

38. The method of claim 27 wherein the metal ions comprise magnesium.

39. The method of claim 23 wherein the PCR reagent is encapsulated by the polylactone matrix.

40. The method of claim 23 wherein the PCR reagent is micro-encapsulated by the polylactone matrix.

41. A kit for hot-start PCR comprising:
a material comprising a polylactone matrix and a first PCR reagent;
wherein the polylactone matrix substantially sequesters the first PCR reagent from participation in a polymerase chain reaction at ambient temperature.

42. The kit of claim 41 wherein the polylactone matrix comprises a polylactone selected from the group consisting of poly(β-propiolactone), poly(β-butyrolactone), poly(γ-butyrolactone), poly(α-methyl-γ-butyrolactone), poly(γ-methyl-γ-hexanolactone), poly(γ-valerolactone), poly(γ-caprolactone), poly(δ-valerolactone), poly(ε-caprolactone), poly(η-heptanolactone), and combinations thereof.

43. The kit of claim 41 wherein the polylactone matrix comprises a polylactone selected from the group consisting of poly(β-propiolactone), poly(γ-butyrolactone), poly(δ-valerolactone), poly(ε-caprolactone), poly(η-heptanolactone), and combinations thereof.

44. The kit of claim 41 wherein the polylactone matrix comprises poly(ε-caprolactone).

45. The kit of claim 41 wherein the first PCR reagent is selected from the group consisting of oligonucleotides, deoxynucleoside triphosphates, polymerase enzymes, metal ions, and combinations thereof.

46. The kit of claim 41 wherein the first PCR reagent comprises an oligonucleotide.

47. The kit of claim 46 wherein the oligonucleotide is selected from the group consisting of sense primers, antisense primers, and a combination thereof.

48. The kit of claim 46 further comprising a second PCR reagent selected from the group consisting of deoxynucleoside triphosphates, polymerase enzymes, metal ions, and combinations thereof.

49. The kit of claim 41 wherein the first PCR reagent comprises a polymerase enzyme.

50. The kit of claim 49 wherein the polymerase enzyme comprises a heat-stable DNA polymerase.

51. The kit of claim 50 wherein the heat-stable DNA polymerase comprises a Taq DNA polymerase.

52. The kit of claim 49 further comprising a second PCR reagent selected from the group consisting of oligonucleotides, deoxynucleoside triphosphates, metal ions, and combinations thereof.

53. The kit of claim 41 wherein the first PCR reagent comprises a metal ion.

54. The kit of claim 53 wherein the metal ion comprises magnesium.

55. The kit of claim 53 further comprising a second PCR reagent selected from the group consisting of oligonucleotides, deoxynucleoside triphosphates, polymerase enzymes, and combinations thereof.
